(19) United States
(12) Patent Application Publication
(10) Pub. No.: US 2009/0170788 A1
(43) Pub. Date: Jul. 2, 2009

(54) NOVEL USE OF 1, 2, 3, 4, 6-PENTA-O-GALLOYL-BETA-D-GLUCOSE

(76) Inventor: Dong-Heon Shin, Seoul (KR)

Correspondence Address: JHK LAW, P.O. BOX 1078, LA CANADA, CA, 91012-1078, US

(21) Appl. No.: 12/161,164

(22) PCT Filed: Jan. 16, 2007
(86) PCT No.: PCT/KR2007/000266

§ 371 Date: Oct. 1, 2008

(30) Foreign Application Priority Data
Jan. 16, 2006 (KR) 10-2006-0004403

(51) Int. Cl. A61K 31/7028; A61P 17/06

(52) U.S. Cl. 514/27

(57) ABSTRACT

The present invention relates to novel use of 1,2,3,4,6-penta-O-galloyl-beta-D-glucose. More particularly, the present invention relates to a pharmaceutical composition and cosmetic composition for preventing and treating skin disease, comprising 1,2,3,4,6-penta-O-galloyl-beta-D-glucose represented by formula 1 as an active ingredient. Also, the invention relates to a method for preventing and treating skin disease using 1,2,3,4,6-penta-O-galloyl-beta-D-glucose, and to the use of 1,2,3,4,6-penta-O-galloyl-beta-D-glucose.

### TECHNICAL FIELD

The present invention relates to novel use of 1,2,3,4,6-penta-O-galloyl-beta-D-glucose. More particularly, the present invention relates to a pharmaceutical composition and cosmetic composition for preventing and treating skin disease, comprising 1,2,3,4,6-penta-O-galloyl-beta-D-glucose as an active ingredient. Also, it relates to a method for preventing and treating skin disease using 1,2,3,4,6-penta-O-galloyl-beta-D-glucose and to the use of 1,2,3,4,6-penta-O-galloyl-beta-D-glucose.

### BACKGROUND ART

Skin diseases refers to all abnormal symptoms appearing on the skin of animals, as well as human beings. Since the skin covers the surface of the body, it is frequently exposed to external stimuli, such as UV light, or various pathogens, and is strongly influenced by the body. Also, it is influenced by pathological changes, such as congenital abnormalities, inflammations, benign and malignant tumors, hormones, traumatic injuries, and degeneration, and thus skin diseases are induced by various causes.

UV light from sunlight holds a small portion of the light reaching the globe, but acts as a factor causing erythema, pigmentation, wrinkles and most of skin cancers. It is known that damages to skin cells and tissues by UV light are induced mainly by highly reactive oxygens, which deteriorate an antioxidant defense system consisting of antioxidant enzymes and non-enzymatic antioxidants to cause symptoms, such as a reduction in firmness, wrinkle formation, epidermal thickening, and collagen degradation, thus causing skin diseases, such as photoaging (Scharffetter-Kochanek et al., Exp Gerontol, 35(3):307-316, 2000) or skin cancer (Hurza and Pentland, J Invest Dermatol, 100:35 S-41S, 1993; Brenneisen P et al., Ann NY Acad Sci 973:3143, 2002; Wlascheck M et al., J Phtochem Photobiol B 63:41-51, 2001). In order to prevent or treat skin disease caused by UV light, it is required to construct a system capable of activating enzymes, which act mainly in vivo and on the skin.

Inflammatory skin disease refers to a disease that involves a series of clinical signs and symptoms, such as itching, edema, erythema and stripping, due to various stimulating factors which cause a series of inflammatory reactions in the skin epidermis. Known inflammatory skin diseases include atopic dermatitis, contact dermatitis, seborrhoic dermatitis and acne.

For the treatment of the inflammatory skin diseases, antihistamine agents, vitamin ointments and adrenal cortical hormones have been used to date. However, such drugs mostly have temporary effects and often show severe side effects.

Tannins are compounds widely distributed in plants and are polymer substances (molecular weight: 600-2000) having complex structures, which consist of various polyphenols, but not single compounds. Tannins are classified, according to characteristics, into hydrolysable tannins, which are easily hydrolyzed by acids or enzymes, and condensable tannins, which are relatively stable against decomposition. The hydrolysable tannins and the condensation tannins are very different from each other with respect to not only decomposition pattern, but also structure and nature (Hagerman A E, Tannin Chemistry. Department of chemistry and biochemistry, Miami University, USA, p 35, 2002). The hydrolysable tannins are easily hydrolyzed to produce sugar and phenolic acid and have a hydroxyl group (—OH), a carboxyl group (—COOH) and their esters (RCOOR) as reactive groups. Also, they are contained in gallnuts, acorns, pomegranates, Geranium nepalense subsp. Thunbergii and the like. Derivatives thereof include 1,2,3,4,6-penta-O-galloyl-beta-D-glucose, 1,2,3,6-tetragalloyl glucose, acetannin and the like. On the other hand, the condensable tannins are not hydrolyzed in the absence of sugar, and are formed of carbon-carbon bonds of catechin and epicatechin. They have only a hydroxyl group (—OH) as a reactive group and are contained in puckery persimmons, tea leaves, Mimosa and the like. Also, derivatives thereof include epigallocatechin-3-gallate (EGCG), delphinidin, luteolinidin, apigeninidin and the like.

Recently, green tea extracts have been found to be effective for skin beauty, and it was reported that the active ingredients thereof was catechin compounds (JUNG Jin-Ho, Sixth International Green Tea Symposium, 2001, Seoul). Also, it was reported that EGCG, which is a condensable tannin derivative and the main component of green tea catechin, had the effect of inhibiting skin aging, when the skin was treated with the EGCG (Salah N et al. Arch Biochem Biophys. 322:339-346, 1995; Kim S Y et al. Arch Pharm Res. 28:784-90, 2005; Lee J H et al. J Dermatol Sci. 2005). Moreover, Korean Patent No. 10-0439590 discloses that cosmetics containing tannin extracted from green tea had effects on the treatment of wrinkles and the inhibition of erythema and skin hardening. However, the preventive or therapeutic effect of hydrolysable tannins on skin diseases is still not reported.

### DISCLOSURE OF THE INVENTION

Accordingly, the present inventors have conducted studies for many years to develop a composition capable of preventing or treating skin diseases and, as a result, found that 1,2,3,4,6-penta-O-galloyl-beta-D-glucose, a hydrolysable tannin derivative, has an activity of preventing or treating skin diseases by inhibiting a reduction in type I collagen, an increase in MMP-1 expression, an increase in COX-2 expression, wrinkle formation, epidermal thickening and collagen degradation, which are induced by UV light, thereby completing the present invention.

An objection of the present invention is to provide the novel use of 1,2,3,4,6-penta-O-galloyl-beta-D-glucose.

To achieve the above objection, in one aspect, the present invention provides a pharmaceutical composition for preventing and treating skin disease, comprising 1,2,3,4,6-penta-O-galloyl-beta-D-glucose or pharmaceutically acceptable salt thereof as an active ingredient.

In another aspect, the present invention provides a cosmetic composition for preventing or improving skin disease, comprising 1,2,3,4,6-penta-O-galloyl-beta-D-glucose or salt thereof.

In still another aspect, the present invention provides a method for treating or preventing skin disease, comprising administrating to a subject in need thereof an effective amount of 1,2,3,4,6-penta-O-galloyl-beta-D-glucose or pharmaceutically acceptable salt thereof.

In still another aspect, the present invention provides the use of 1,2,3,4,6-penta-O-galloyl-beta-D-glucose or pharmaceutically acceptable salt thereof for the manufacture of a pharmaceutical agent for treating skin disease.

In still another aspect, the present invention provides the use of 1,2,3,4,6-penta-O-galloyl-beta-D-glucose or salt thereof for the manufacture of a cosmetic agent of preventing or improving skin disease.

Hereinafter, the present invention will be described in detail.

1,2,3,4,6-penta-O-galloyl-beta-D-glucose of the present invention is represented by formula 1 below:

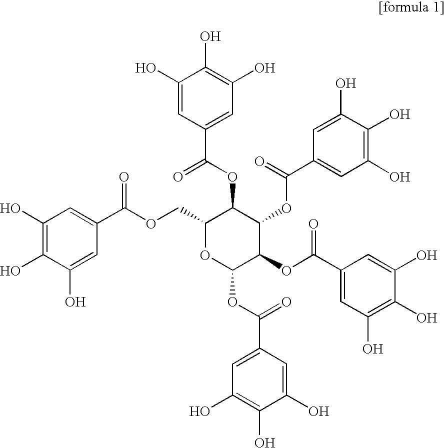

1,2,3,4,6-penta-O-galloyl-beta-D-glucose of the present invention may be used for itself or in the form of a salt, preferably a pharmaceutically acceptable salt. Preferably, the salt is an acid-addition salt formed using a pharmaceutically acceptable free acid. The free acid used in the present invention may be selected from among organic acids and inorganic acids. The organic acids include, but are not limited to, citric acid, acetic acid, lactic acid, tartar acid, maleic acid, fumaric acid, formic acid, propionic acid, oxalic acid, trifluoroacetic acid, benzoic acid, gluconic acid, m-sulfonic acid, glycolic acid, succinic acid, 4-toluene sulfonic acid, glutamic acid and aspartic acid. Also, the inorganic acids include, but are not limited to, hydrochloric acid, bromic acid, sulfuric acid and phosphoric acid.

1,2,3,4,6-penta-O-galloyl-beta-D-glucose of the present invention can be obtained according to any conventional method for extracting and isolating substance. Preferably, 1,2,3,4,6-penta-O-galloyl-beta-D-glucose of the present invention can be obtained from tannic acid by conventional methanolysis and normal-phase or reverse-phase chromatography. Also, 1,2,3,4,6-penta-O-galloyl-beta-D-glucose of the present invention can be obtained from peony by a conventional extracting method and chromatography (Korea Patent Application Publication No. 2003-0075947), and can be chemically manufactured using any known synthetic method.

1,2,3,4,6-penta-O-galloyl-beta-D-glucose according to the present invention can prevent and treat skin diseases by inhibiting various substances that mediate skin diseases.

The main causes of skin diseases induced by UV light are that the active oxygen species formed by UV light transmit signals to a signaling system to stimulate the synthesis of MMP-1 (matrix metalloproteinase-1), an enzyme of degrading skin's intercellular substances such as collagen (Angel, P. et al. Cell, 49:729-739, 1987; Devary, Y., et al. Science, 261:1442-1445, 1993). MMP-1 degrades collagen to cause skin disease symptoms, such as a reduction in skin firmness, epidermal thickening of the skin, and wrinkle formation. Also, when a collagen structure needs to be degraded, MMP-1 is expressed in a metabolic process, so that it regulates a collagen metabolism performing an important role in wound repair, tissue resorption, embryonic development, vascular formation and cell migration and is involved in the degradation of the basement membrane in rheumatism and cancer cell metastasis (Bresalier R. S. et. al, Cancer Res., 47:1398-1406, 1987). Thus, if an increase in MMP-1 expression, caused by UV radiation, is inhibited, a reduction in skin firmness, skin's epidermal thickening, and wrinkle formation, can be prevented, and skin diseases caused by UV radiation can be prevented and treated.

In in vitro and in vivo tests, 1,2,3,4,6-penta-O-galloyl-beta-D-glucose according to the present invention inhibits an increase in MMP-1 expression, caused by UV radiation.

It was observed that, in in vitro tests, 1,2,3,4,6-penta-O-galloyl-beta-D-glucose of the present invention increases type I procollagen expression reduced by UV radiation, and reduces MMP-1 expression increased by UV radiation. Herein, since the skin protective effect of EGCG, which is a condensable tannin derivative and the main component of green tea catechin, is known in the art, the effect thereof was also examined. Treatment with 1,2,3,4,6-penta-O-galloyl-beta-D-glucose showed a marked increase in the expression of type I procollagen compared to the case of treatment with EGCG. Also, the inhibitory effect of 1,2,3,4,6-penta-O-galloyl-beta-D-glucose on MMP-1 was much more superior to the MMP-1 inhibitory effect of EGCG, reported in Korean Patent No. 10-0439590 (see FIGS. 2 and 3).

In in vivo tests, 1,2,3,4,6-penta-O-galloyl-beta-D-glucose inhibited a reduction in type I procollagen, and the expression of MMP-1, caused by UV light (see 8 and 9).

COX-2 is a substance involved in inflammatory reactions in the body, is largely expressed in skin cells by external stimuli such as UV light, converts arachidonic acid in the cellular membrane into prostaglandin to stimulate skin cell damage, and is overexpressed in damaged skin tissue. Thus, when the expression of COX-2 is inhibited, inflammatory skin diseases can be prevented.

Also, it was reported that the overexpression of COX-2 was shown in various malignant tumors, inhibited apoptosis and increased the penetration ability of tumor-inducing cells (Dempke et al. J. Can. Res. Clin. Oncol. 127:411-17, 2001). Thus, when the expression of COX-2 is inhibited, skin cancer can be prevented.

In in vitro tests, 1,2,3,4,6-penta-O-galloyl-beta-D-glucose of the present invention showed the effect of inhibiting an increased expression of COX-2, caused by UV radiation (see FIG. 4).

In in vivo tests, 1,2,3,4,6-penta-O-galloyl-beta-D-glucose of the present invention showed the effect of promoting collagen formation, when skin tissue having reduced collagen due to UV radiation was treated with the 1,2,3,4,6-penta-O-galloyl-beta-D-glucose (see FIG. 7).

In addition, 1,2,3,4,6-penta-O-galloyl-beta-D-glucose of the present invention showed the effect of inhibiting epidermal thickening and wrinkle formation caused by UV light (see FIGS. 5 and 6).

Accordingly, the present invention provides a pharmaceutical composition for preventing and treating skin disease, comprising 1,2,3,4,6-penta-O-galloyl-beta-D-glucose or pharmaceutically acceptable salt thereof as an active ingredient.

Said skin disease means skin disease by ultraviolet radiation or inflammatory skin disease.

Said “skin disease by ultraviolet radiation” may include, but are not limited to, photo aging or skin cancer.

Also, said inflammatory skin disease may include, but are not limited to, skin inflammation, acute and chronic eczema, contact dermatitis, atopic dermatitis, seborrheic dermatitis, lichen simplex chronicus, intertrigo, dermatitis exfoliativa, papular urticaria, psoriasis, solar dermatitis and acne.

The pharmaceutical composition of the present invention may comprise 1,2,3,4,6-penta-O-galloyl-beta-D-glucose alone or may further comprise at least one pharmaceutically acceptable carrier, excipient or diluent. As used herein, the term “pharmaceutically acceptable” means that a composition is physiologically acceptable, and when administered to human beings it does not cause allergic reactions or similar reactions.

The pharmaceutical composition of the present invention for preventing and treating skin disease may be administered to mammals by any routes according to any method known in the art. For example, it may be administered by oral or parenteral routes. Examples of parenteral routes are, but are not limited to, intracutaneous, subcutaneous, intravenous, intramuscular or Intraperitoneal Administration. The term ‘intracutaneous administration’ as used herein means that the pharmaceutical composition of the present invention is administrated in cell or skin, thereby transmitting active ingredients, which is comprised in the pharmaceutical composition for preventing or treating skin disease, to the inside of skin. For example, the pharmaceutical composition of the present invention may be formulated into a formulation for injection, which is administered by lightly pricking the skin with 30 gauge injection needle. Otherwise, such formulations for injection may be applied directly onto the skin.

The pharmaceutical composition of the present invention may be formulated into various forms for oral or parenteral administration according to administration routes.

In the case of an oral formulation, the composition of the present invention may be formulated into powder, granule, tablet, pill, troche, capsule, elixir, gel, syrup, slurry, suspension and the like by conventional methods known to one skilled in the art. For example, the oral preparations may be obtained as tablets or sugar-coated tablets by blending the active ingredient with a solid excipient, crushing the blend, adding suitable adjuvants and then processing the mixture into a granular mixture. Examples of suitable excipients may include sugars including lactose, dextrose, sucrose, sorbitol, mannitol, xylitol, erythritol and maltitol; starches including corn starch, wheat starch, rice starch and potato starch; celluloses including cellulose, methyl cellulose, sodium carboxymethylcellulose and hydroxypropylmethyl cellulose; and fillers including zelatin and polyvinylpyrrolidone. Also, the pharmaceutical composition of the present invention may, if necessary, contain a disintegrant, such as crosslinked polyvinylpyrrolidone, agar, alginic acid or sodium alginate. Furthermore, the pharmaceutical composition may further comprise an anticoagulant, a lubricant, a wetting agent, a perfumery, an emulsifier, and a preservative.

In case of the parenteral preparations, they can be formulated in the form of injections, creams, lotions, external ointments, oils, moisturizers, gels, aerosols, and nasal inhalers by any method known in the art. These preparations are described in the following formulary known to all pharmaceutical chemists: Remington's Pharmaceutical Science, 15th Edition, 1975, Mack Publishing Company, Easton, Pa. 18042, Chapter 87: Blaug, Seymour.

The total effective amount of 1,2,3,4,6-penta-O-galloyl-beta-D-glucose of the present invention can be administered to a subject as a single dose, or can be administered using a fractionated treatment protocol, in which the multiple doses are administered over a more prolonged period of time.

The content of the active ingredient in the pharmaceutical composition of the present invention can be varied depending on the severity of skin disease. Preferably, the total dose of 1,2,3,4,6-penta-O-galloyl-beta-D-glucose of the present invention is 0.01 μg/kg/day to 1,000 mg/kg/day, and more preferably 0.1 μg/kg/day to 100 mg/kg/day. However, one skilled in the art would know that the concentration of 1,2,3,4,6-penta-O-galloyl-beta-D-glucose of the present invention required to obtain an effective dose in a subject depends on many factors including the age, bodyweight, health condition, disease severity, diet and excretion of the subject, the route of administration, the number of treatments to be administered, and so forth. In view of these factors, any person skilled in the art can determine the suitable effective dose of 1,2,3,4,6-penta-O-galloyl-beta-D-glucose for preventing or treating skin diseases. No particular limitation is imposed on the formulation, administration route and administration mode of the pharmaceutical composition according to the present invention, as long as the composition shows the effects of the present invention.

Also, from the above test results, the present inventors have found that 1,2,3,4,6-penta-O-galloyl-beta-D-glucose has the effect of inhibiting wrinkle formation, epidermal thickening, collagen degradation, a reduction in type I procollagen, an increase in MMP-1 expression, and an increase in COX-2 expression, which are caused by UV light. According to this finding, we prepared a cosmetic composition containing 1,2,3,4,6-penta-O-galloyl-beta-D-glucose. The cosmetic composition comprises, in addition to 1,2,3,4,6-penta-O-galloyl-beta-D-glucose of the present invention, at least one excipient and additive generally used in the manufacturing field of cosmetic compositions, and can be easily prepared according to any method known in the art.

More specifically, the cosmetic composition according to the present invention contains, as an active ingredient, 1,2,3,4,6-penta-O-galloyl-beta-D-glucose of the present invention, together with dermatologically acceptable excipients, and can be prepared in the form of base cosmetic compositions (e.g., skin lotion, cream, essence, facial cleaners such as cleansing foam and cleansing water, pack, body oil, etc.), color cosmetic compositions (e.g., foundation, lipstick, mascara, makeup base, etc.), hair product compositions (e.g., shampoo, hair conditioner, hair gel, etc.), and soap. The excipients may include, but are not limited to, for example, skin softeners, skin penetration enhancers, coloring agents, aromatics, emulsifying agents, thickening agents, and solvents. Also, the composition may additionally comprise perfume, a pigment, a sterilizing agent, an antioxidant, a preservative and a moisturizing agent, and may comprise a thickener, an inorganic salt, a synthetic polymer substance and the like for improving the physical properties of the composition.

For example, in the case of preparing a facial cleaner and soap, which contain 1,2,3,4,6-penta-O-galloyl-beta-D-glucose of the present invention, they can be easily prepared by adding 1,2,3,4,6-penta-O-galloyl-beta-D-glucose to a conventional facial cleaner or soap base. In the case of preparing cream, it can be prepared by adding 1,2,3,4,6-penta-O-galloyl-beta-D-glucose to a general oil-in-water (O/W) cream base. Also, it is possible to add perfume, a chelating agent, a pigment, an antioxidant, a preservative and the like, and synthetic or natural materials for improving physical properties, including proteins, minerals and vitamins.

The content of 1,2,3,4,6-penta-O-galloyl-beta-D-glucose in the inventive cosmetic composition may be 0.0001-50 wt %, and preferably 0.01-10 wt %, based on the total weight of the cosmetic composition.

On the basis of the effect of 1,2,3,4,6-penta-O-galloyl-beta-D-glucose of the present invention as mentioned above, the present invention provides a method for treating or preventing skin disease comprising administrating to a subject in need thereof an effective amount of 1,2,3,4,6-penta-O-galloyl-beta-D-glucose or pharmaceutically acceptable salt thereof.

The term “effective amount” as used herein means an amount of 1,2,3,4,6-penta-O-galloyl-beta-D-glucose of the present invention showing the effect of preventing or treating skin diseases in a subject in need thereof. The term “subject” as used herein means mammals, especially mammals, including human beings. The subject may be a patient in need of treatment.

1,2,3,4,6-penta-O-galloyl-beta-D-glucose of the present invention can be administered until the desired effects among the above-described effects can be obtained. Also, it can be administered through various routes according to any method known in the art. Specifically, it can be administered through oral or parenteral routes, for example, oral, intramuscular, intravenous, intradermal, intraarterial, intraosseous, intrathecal, intraperitoneal, intranasal, intravaginal, intrarectal, sublingual or subcutaneous routes, or can be administered into gastrointestinal tracts, mucosa or respiratory organs.

Also, the present invention provides the use of 1,2,3,4,6-penta-O-galloyl-beta-D-glucose or pharmaceutically acceptable salt thereof for the manufacture of an agent of treating skin disease. Furthermore, the present invention provides the use of 1,2,3,4,6-penta-O-galloyl-beta-D-glucose or salt thereof for the manufacture of a cosmetic agent of preventing or improving skin diseases. Skin disease, 1,2,3,4,6-penta-O-galloyl-beta-D-glucose of the present invention and the effects thereof are the same as described above.

### BRIEF DESCRIPTION OF THE DRAWINGS

FIG. 1 shows the results of high-speed liquid chromatography of an ethyl acetate fraction.

FIG. 2 is a graph showing the effects of 1,2,3,4,6-penta-O-galloyl-beta-D-glucose (PGG) on the expression patterns of type I procollagen (A) and MMP-1 (B) caused by UV A radiation in human skin fibroblasts.

* indicates significance at a reliability of 95%.

FIG. 3 is a graph showing the effects of 1,2,3,4,6-penta-O-galloyl-beta-D-glucose EGG) on the expression patterns of type I procollagen (A) and MMP-1 (B) caused by UV B radiation in human skin fibroblasts.

* indicates significance at a reliability of 95%.

FIG. 4 shows the results of Western blot analysis for the effect of 1,2,3,4,6-penta-O-galloyl-beta-D-glucose (PGG) on the expression pattern of a COX-2 protein caused by UV B radiation in human keratinocytes.

FIG. 5 shows the results for the inhibitory effect of 1,2,3,4,6-penta-O-galloyl-beta-D-glucose (PGG) on skin wrinkle formation caused by UV radiation, in mouse model.

A: a photograph of the skin of an untreated group treated with neither UV light nor 1,2,3,4,6-penta-O-galloyl-beta-D-glucose (PGG);

B: a photograph of the skin of a group treated with UV light alone;

C: a photograph of the skin of a test group treated with both UV light and 1,2,3,4,6-penta-O-galloyl-beta-D-glucose;

D: a skin replica of an untreated group treated with neither UV light nor 1,2,3,4,6-penta-O-galloyl-beta-D-glucose (PGG);

E: a skin replica of a group treated with UV light alone; and

F: a skin replica of a test group treated with both UV light and 1,2,3,4,6-penta-O-galloyl-beta-D-glucose.

FIG. 6 shows the results for the inhibitory effect of 1,2,3,4,6-penta-O-galloyl-beta-D-glucose on skin's epidermal thickening induced by UV radiation, in mouse model.

▴: thin epidermis, ↑: panniculus carnosus, *: keratinizing cysts; □: 200× amplification;

A: a photograph of the skin of an untreated group treated with neither UV light nor 1,2,3,4,6-penta-O-galloyl-beta-D-glucose;

B: a photograph of the skin of a group treated with UV light alone; and

C: a photograph of the skin of a test group treated with both UV light and 1,2,3,4,6-penta-O-galloyl-beta-D-glucose.

FIG. 7 shows the results for the collagen formation promoting effect of 1,2,3,4,6-penta-O-galloyl-beta-D-glucose against collagen degradation induced by UV radiation, in mouse model.

▴: epidermis; †: collagen;

A: a photograph of the skin of an untreated group treated with neither UV light nor 1,2,3,4,6-penta-O-galloyl-beta-D-glucose;

B: a photograph of the skin of a group treated with UV light alone; and

C: a photograph of the skin of a test group treated with both UV light and 1,2,3,4,6-penta-O-galloyl-beta-D-glucose.

FIG. 8 shows the results for the effect of 1,2,3,4,6-penta-O-galloyl-beta-D-glucose on the promotion of expression of type I procollagen showing decreased expression due to UV radiation in mouse model.

□: 400× enlarged; ▴: type I procollagen;

A: a photograph of the skin of an untreated group treated with neither UV light nor 1,2,3,4,6-penta-O-galloyl-beta-D-glucose;

B: a photograph of the skin of a group treated with UV light alone; and

C: a photograph of the skin of a test group treated with both UV light and 1,2,3,4,6-penta-O-galloyl-beta-D-glucose.

FIG. 9 shows the results for the inhibitory effect of 1,2,3,4,6-penta-O-galloyl-beta-D-glucose on MMP-1 showing increased expression due to UV radiation in mouse model.

▴: MMP-1.

A: a photograph of the skin of an untreated group treated with neither UV light nor 1,2,3,4,6-penta-O-galloyl-beta-D-glucose;

B: a photograph of the skin of a group treated with UV light alone; and

C: a photograph of the skin of a test group treated with both UV light and 1,2,3,4,6-penta-O-galloyl-beta-D-glucose.

### BEST MODE FOR CARRYING OUT THE INVENTION

Hereinafter, the present invention will be described in detail with reference to examples. It is to be understood, however, that these examples are illustrative only and the scope of the present invention is not limited thereto.

### Example 1

### Isolation of 1,2,3,4,6-penta-O-galloyl-beta-D-glucose

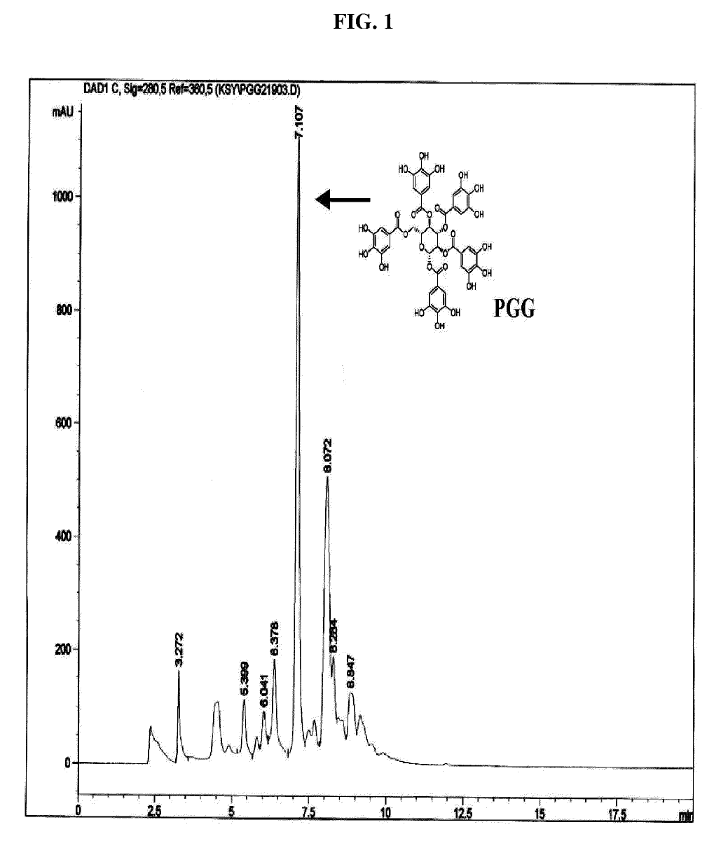

1,2,3,4,6-penta-O-galloyl-beta-D-glucose was prepared from tannic acid by methanolysis. A commercially available tannic acid (Sigma, Mo., USA) was used as a reaction material. 10 g of tannic acid was subjected to a methanolysis reaction in a mixture of acetate buffer and methanol (3:7 (v/v)) at a temperature of 55-65° C. for 15-18 hours. The reaction solution was adjusted to a weakly acidic condition of pH 6.0 and concentrated in a vacuum at a temperature of less than 30° C. to remove methanol. The volume of the concentrate was adjusted with water, and the aqueous solution was fractionated using ethyl ether according to polarity. Then, the ethyl ether fraction was fractionated using ethyl acetate according to polarity. The ethyl acetate fraction was concentrated using a vacuum concentrator at a temperature of less than 30° C., and the residue layer was freeze-dried using a freeze-dryer. Then, the degree of methanolysis of each of the solvent fractions was analyzed using high-performance liquid chromatography and, as a result, it was seen that 1,2,3,4,6-penta-O-galloyl-beta-D-glucose among tannin derivatives was contained predominately in the ethyl acetate fraction (see FIG. 1).

300 mg of the ethyl acetate fraction was injected into a reverse phase column (1.0×300 mm, Waters) of high performance liquid chromatography (GILSON) (1.0×300 mm, Waters), and 1.5 ml of each of eluates was obtained in a test tube while changing the concentrations of 0.1% trifluoracetic acid-containing acetonitrile and 0.1% trifluoracetic acid-containing water (20:80->40:60) as mobile phase solvents. The eluant containing 1,2,3,4,6-penta-O-galloyl-beta-D-glucose was concentrated in vacuum with a vacuum concentrator (speed vac) to isolate 10 mg of an pure substance. The isolated substance was analyzed by mass spectrometry and, as a result, it was found that the isolated substance was 1,2,3,4,6-penta-O-galloyl-beta-D-glucose having a molecular weight of 940 (date not shown).

Also, in order to increase the production yield for commercial use of the isolated 1,2,3,4,6-penta-O-galloyl-beta-D-glucose, 3.0 g (dry weight) of the ethyl acetate fraction determined to contain 1,2,3,4,6-penta-O-galloyl-beta-D-glucose predominately therein was mixed with the same amount of water in the process during which the tannic acid reaction solution subjected to methanolysis was fractionated with the solvent. Then, the solution was concentrated in a vacuum, and the ethyl acetate fraction was forced to dissolve in water and recrystallized at refrigerating temperature, thus obtaining a white crystal. The obtained crystal was repeatedly washed and recrystallized using a water-soluble alcohol solution, thus isolating 200 mg of 1,2,3,4,6-penta-O-galloyl-beta-D-glucose.

The 1,2,3,4,6-penta-O-galloyl-beta-D-glucose isolated from tannic acid was dissolved in DMSO at a concentration of 100 mg/ml and stored at −20° C. before use.

### Example 2

### Inhibitory Effects of 1,2,3,4,6-penta-O-galloyl-beta-D-glucose on Reduction in type I Collagen and Increase in MMP-1, Caused by UV Light, in Cells In Vitro

Human normal dermal fibroblasts, which were isolated and cultured from the skin dermal layer cut from infant phimosis, were seeded into a 6-well plate at a density of 1×10⁵ cells/well, and then cultured in 10% fetal bovine serum-containing DMEM (Dulbecco's modified Eagle's medium) for 24 hours. After the culture, the cells were washed three times with phosphate buffered saline (PBS) and then irradiated with UV A and -w B in the following manner in order to induce skin aging.

UV A was irradiated using a UV A lamp (Sankyo Denki FL8BL lamp, Sankyo Denki Co., Japan) to a total energy dose of 50 mJ/cm² at a rate of 27 mW/cm²/sec, and UV B was irradiated using a UV B lamp (Sankyo Denki G9T5E lamp, Sankyo Denki Co., Japan) to a total energy dose of 170 mJ/cm² at a rate of 1.15 mW/cm²/sec. Just after UV irradiation, the cells were washed again with phosphate buffered saline (PBS), and 1,2,3,4,6-penta-O-galloyl-beta-D-glucose was added to a DMEM medium containing fetal bovine serum at concentrations of 1 and 10 μg/ml. The cells were further cultured in the medium for 24 hours. Test groups were divided as follows:

Untreated group: treated with neither UV light nor 1,2,3,4,6-penta-O-galloyl-beta-D-glucose;

Group treated with UV light alone: treated only with UV light;

Control group: treated with UV light and EGCG; and

Test Group: treated with both UV light and 1,2,3,4,6-penta-O-galloyl-beta-D-glucose.

Because EGCG, which is a condensable tannin derivative and the main component of green tea catechin, was reported in the Korean Patent to have effects on the inhibition of MMP-1 expression and the promotion of type I procollagen expression, it was used as a control group. In order to examine if the skin was aged after the culture, the expression levels of skin intercellular matrix component type I procollagen and its degradation enzyme MMP-1 were measured using a type I procollagen ELISA kit (Procollagen Type I C-Peptide EIA kit, TaKaRa, Japan) and a MMP-1 ELISA kit (MMP-1 human, ELISA system, Biotrak Assay, Amersham Biosciences, USA) according to manufacturer's protocols. The concentrations of type I procollagen and MMP-1 in cells were shown as ng of proteins per ml of total cell culture media.

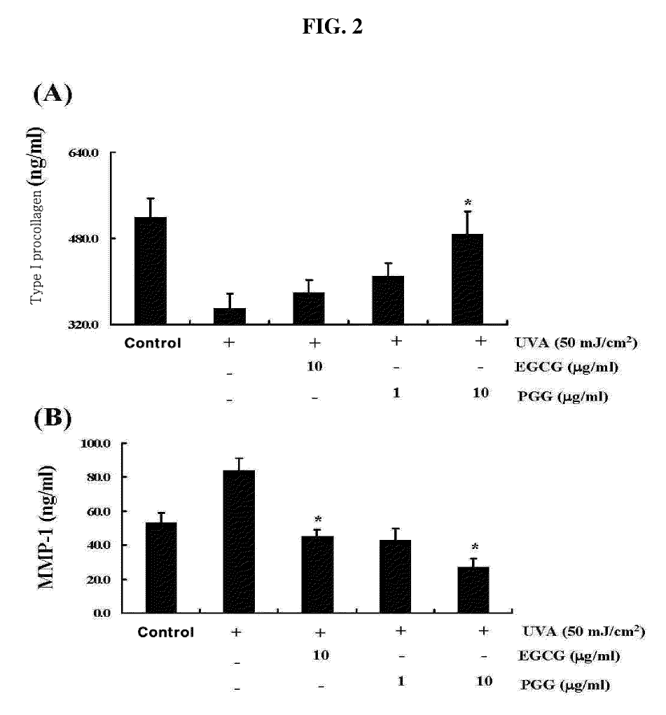

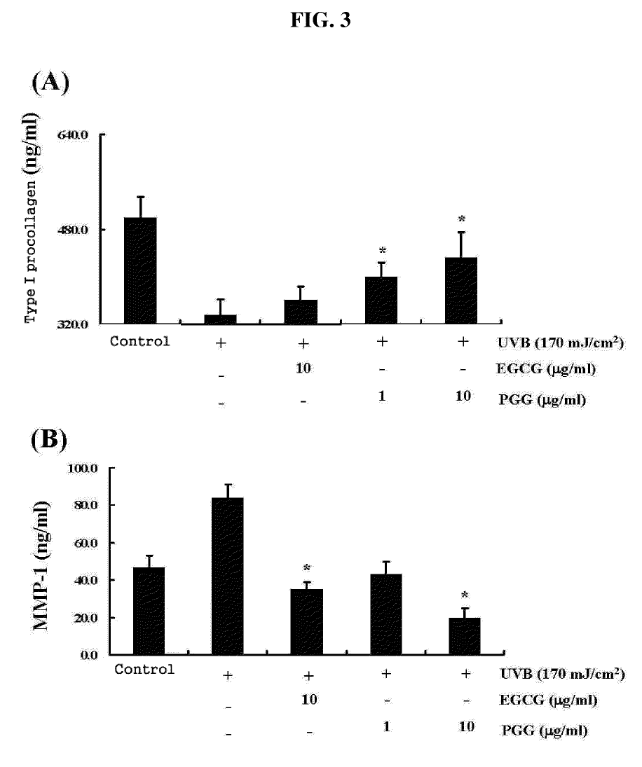

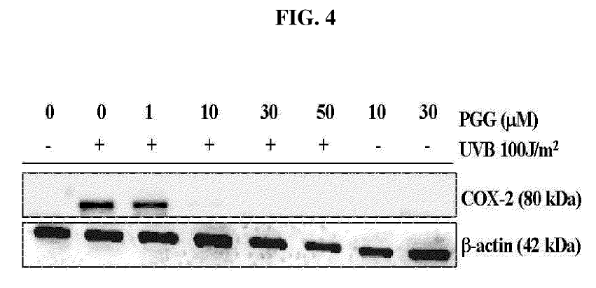

As a result, it could be seen that, in the case where the human normal dermal fibroblasts were irradiated with UV light (group treated with UV light alone), the expression of type I procollagen was decreased, whereas the expression of MMP-1 was increased. Also, an increase in the expression of type I procollagen and a decrease in the expression of MMP-1, caused by treatment with 1,2,3,4,6-penta-O-galloyl-beta-D-glucose, were shown in a concentration-dependent manner. Moreover, the effect of 1,2,3,4,6-penta-O-galloyl-beta-D-glucose was higher than the effect of EGCG, reported in the Korean Patent (see FIGS. 2 and 3).

From the above results, it was found that 1,2,3,4,6-penta-O-galloyl-beta-D-glucose increased the expression of type I procollagen, which was reduced due to UV radiation, and it decreased the expression of MMP-1, which was increased due to UV radiation.

### Example 3

### Inhibitory Effect of 1,2,3,4,6-penta-O-galloyl-beta-D-glucose on Increased COX-2, Caused by UV Light, in Cells In Vitro

Human keratinocytes (HaCaT cell) provided by professor N. Fusenig (German Cancer Research, Germany) were seeded into a 100 mm culture dish to 1.3×10⁶ cells, and then cultured in a DMEM medium containing 10% fetal bovine serum for 24 hours. After the culture, the cells were washed three times with phosphate buffered saline, and then irradiated with UV B in the following manner in order to induce skin aging. UV B was irradiated using an UV B lamp to a total energy dose of 10 mJ/cm² at a rate of 0.6 mW/cm²/sec. Just after the UV irradiation, the cells were washed again with phosphate buffered saline, and 1,2,3,4,6-penta-O-galloyl-beta-D-glucose was added to a DMEM medium containing no fetal bovine serum at concentrations of 1, 10, 30, 50 μM. Then, the cells were further cultured in the medium for 12 hours. Test groups were divided as follows:

Untreated group: treated with neither UV light nor 1,2,3,4,6-penta-O-galloyl-beta-D-glucose;

Group treated with UV light alone: treated only with UV light; and

Test group: treated with both UV light and 1,2,3,4,6-penta-O-galloyl-beta-D-glucose.

After the 12-hr culture, the cells were washed with cold phosphate buffered saline, and then treated with lysis buffer to separate a cell protein. Then, the cell protein was subjected to Western blot analysis using a COX-2 antibody (Santa Cruz Biotch., USA) and a horseradish peroxidase-conjugated secondary antibody (ZYMED Laboratories, USA). As a loading control group, 0-actin was used.

As a result, it could be seen that, when the human keratinocytes were irradiated with UV light, the expression of COX-2 was increased compared to the untreated group. Also, treatment with 1,2,3,4,6-penta-O-galloyl-beta-D-glucose reduced the expression of COX-2 in a concentration-dependent manner (see FIG. 4).

From the above results, it was found that 1,2,3,4,6-penta-O-galloyl-beta-D-glucose reduced the expression of COX-2, which was increased due to UV radiation, in the skin keratinocytes.

### Example 4

### Inhibitory Effect of 1,2,3,4,6-penta-O-galloyl-beta-D-glucose on Wrinkle Formation Caused by UV Light, in Mouse Model

Whether 1,2,3,4,6-penta-O-galloyl-beta-D-glucose has the effect of inhibiting wrinkle formation caused by UV light was examined. First, 7-week-old female hairless mice (albino SKH-1 hairless mice, Hanlim Experimental Animal Co., Suwon, Korea) were divided into the following three groups, each consisting of 10 animals.

Untreated group: treated with neither UV light nor 1,2,3,4,6-penta-O-galloyl-beta-D-glucose;

Group treated with UV light alone: treated with UV light and a composition containing no 1,2,3,4,6-penta-O-galloyl-beta-D-glucose; and

Test group: treated with UV light and a composition containing 1,2,3,4,6-penta-O-galloyl-beta-D-glucose.

The mice of each of the groups were put in an UV box equipped with an UV B lamp, and were irradiated with UV light at a dose of 100 mJ/cm² corresponding to minimal erythemal dose (MED). UV light was irradiated one time a day, three times a week for 4 weeks while increasing the dose of UV light from 100 mJ/cm² at the first week to 200 mJ/cm² at the second week, 300 mJ/cm² at the third week and 400 mJ/cm² at the fourth week so as to adapt the mice to UV light. Following the fourth week, the mice were irradiated with UV light at a dose of 400 mJ/cm² for 6 weeks, and thus the hairless mice were exposed to UV light for a total of 10 weeks, thereby inducing skin photoaging. (Kilgman L H, Clin Dermatol 14:183-195, 1996). In the case of the test group, starting from 4 weeks after UV irradiation, a composition containing 1,2,3,4,6-penta-O-galloyl-beta-D-glucose was dissolved in a propylene glycol solution containing 30% ethanol (7:3 [v/v] propylene glycol:ethanol) and applied to the skin of the mice in an amount of 10 mg/200 μl/4 cm². In the case of the group treated with UV light alone, a composition containing no 1,2,3,4,6-penta-O-galloyl-beta-D-glucose was applied to the mice in an amount of 200 μl/4 cm² in the same manner as the test group. At 24 hours after completion of final UV irradiation at the 10^{th} week, the degree of skin wrinkles in the mice was observed using skin photographs of the mice and a skin replica kit (Courage+Khazaka Electronic, Koln, Germany).

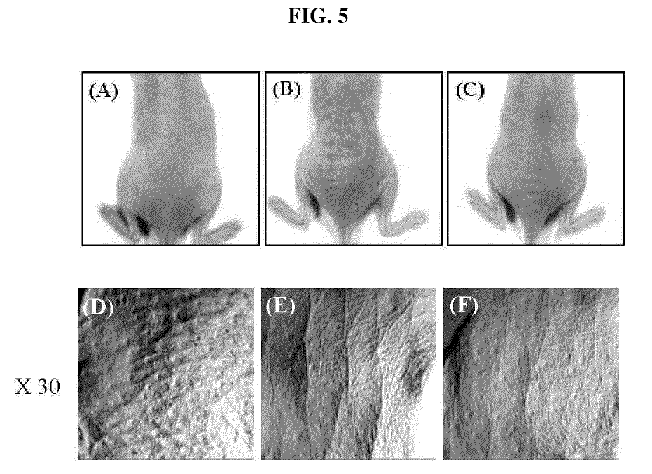

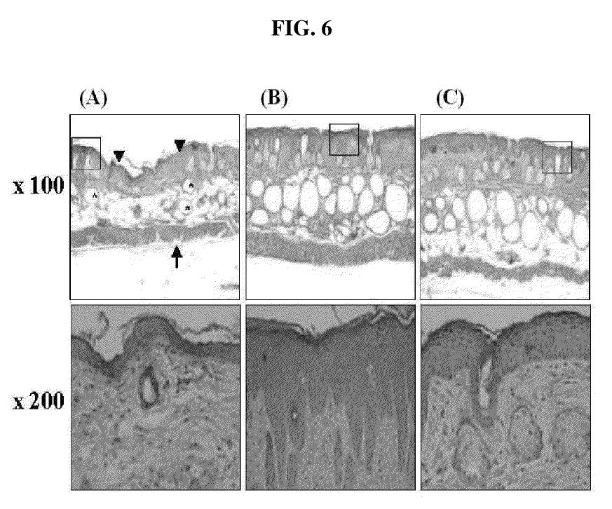

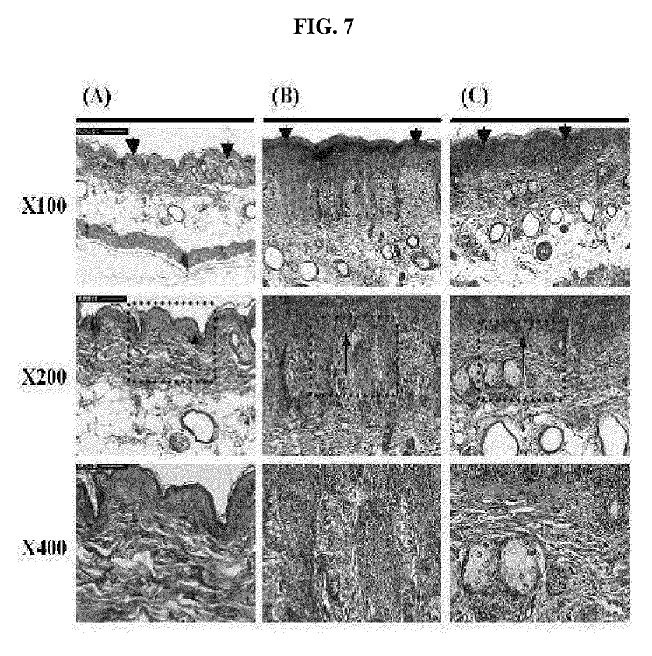

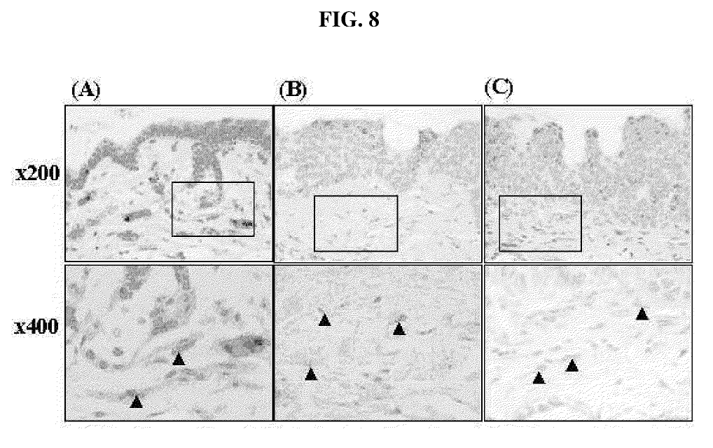

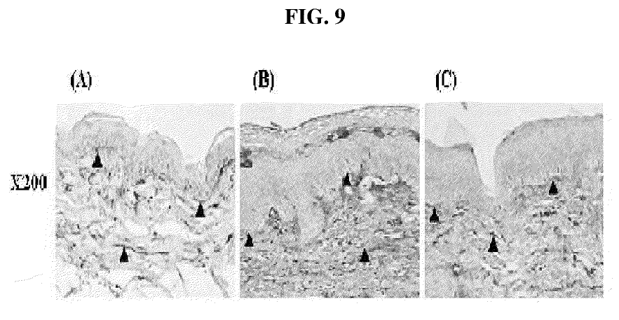

As a result, the frequency of wrinkle formation was increased due to UV irradiation, and treatment with 1,2,3,4,6-penta-O-galloyl-beta-D-glucose reduced the frequency of wrinkle formation (see FIG. 5).

As a result, it was found that 1,2,3,4,6-penta-O-galloyl-beta-D-glucose inhibited the formation of wrinkles.

### Example 5

### Inhibitory Effect of 1,2,3,4,6-penta-O-galloyl-beta-D-glucose on Epidermal Thickening in Mouse Model

Hairless mice were treated with UV light and 1,2,3,4,6-penta-O-galloyl-beta-D-glucose in the same manner as in Example 4, and the skin tissue thereof was extracted. The extracted tissue was immersed in 4% neutral formalin solution for more than 24 hours, and the tissue was washed, dehydrated and fixed according to conventional methods, and then embedded in paraffin to prepare a paraffin block. The paraffin block was sectioned to prepare tissue sections having a thickness of 5 μm. To examine the thickening of the skin epidermal layer through the arrangement and morphological change of cells, the tissue sections were stained with a hematoxylin-eosin (H&E) staining method, in which the tissue sections were deparaffined and rehydrated, and then subjected to progressive staining using Mayer's hematoxylin solution and eosin solution, and the tissue sections were then dehydrated, fixed and sealed. The optical microscopic observation was performed and, as a result, the hematoxylin staining solution stained the nuclei in blue color, and the eosin staining solution stained the cytoplasm in red color.

As a result, the thickening of the skin epidermal layer was significantly higher in the UV-irradiated tissue (group treated with UV light alone) compared to the UV-non-radiated tissue (untreated group). However, in the case of treatment with 1,2,3,4,6-penta-O-galloyl-beta-D-glucose (test group), the thickening of the skin epidermal layer was significantly decreased (see FIG. 6).

As a result, it was found that 1,2,3,4,6-penta-O-galloyl-beta-D-glucose inhibited the skin epidermal layer, induced by UV light.

### Example 6

### Collagen Formation-Promoting Effect of 1,2,3,4,6-penta-O-galloyl-beta-D-glucose in Mouse Model

Hairless mice were treated with UV light and 1,2,3,4,6-penta-O-galloyl-beta-D-glucose in the same manner as in Example 4, and the skin tissue thereof was extracted. To observe the change of collagen among skin's connective tissues damaged by UV light, skin tissue sections were prepared in the same manner as in Example 5. The tissue sections were deparaffined and hydrated, and then subjected to masson trichome staining using three dyes. More specifically, the tissue sections were immersed in a Weigert's iron hematoxylin solution to stain the nuclei, and then immersed in a Biebrich scarlet acid fusion solution to stain the cytoplasm, keratin and muscle fiber of the tissue. Finally, the tissue sections were immersed in a 2% light green solution to stain only collagen, and then were subjected to masson trichome staining, in which the tissue sections were dehydrated, fixed and sealed. Then, the tissue sections were observed with an optical microscope. The cellular nucleus was stained in black, the cytoplasm, keratin and muscular fibers in the tissue sections were stained in red, and the dermal layer was stained in bluish green.

As a result, it could be seen that the thickness of the epithelial layer was much larger in the UV-irradiated tissue (group treated with UV light alone) compared to the UV-non-irradiated tissue (untreated group), and the fragmentation of collagen fiber became thin in the case of the UV-irradiated group. Meanwhile, in the case of treatment with 1,2,3,4,6-penta-O-galloyl-beta-D-glucose (test group), the amount of collagen was increased compared to the group treated with UV light alone, and the amount of damaged collagen was decreased (see FIG. 7).

From the above results, it was found that 1,2,3,4,6-penta-O-galloyl-beta-D-glucose showed the effect of inhibiting collagen damage induced by UV light, and the effect of promoting collagen formation.

### Example 7

### Inhibitory Effects of 1,2,3,4,6-penta-O-galloyl-beta-D-glucose on Reduction In Type I Collagen and Increase in MMP-1, Caused by UV Light, in Mouse Model

In order to further confirm the effect (demonstrated in Example 2 above) of 1,2,3,4,6-penta-O-galloyl-beta-D-glucose in mouse model, hairless mice were treated with UV light and 1,2,3,4,6-penta-O-galloyl-beta-D-glucose in the same manner as in Example 4. The skin tissue obtained through extraction after the treatment was sectioned in the same manner as in Example 5. The tissue sections were deparaffined, hydrated, and then subjected to immunohistochemistry according to the method of Griffiths (Grittiths et al. New Eng J. Med. 329:530-535, 1993). The expressions of type I procollagen and MMP-1 were observed using a rat anti-human type I procollagen antibody (Chemicon, USA) and a rabbit anti-human MMP-1 antibody by means of an optical microscope.

As a result, in the case of irradiation with UV light only (group treated with UV light alone), the expression of type I procollagen was decreased, but in the case of treatment with 1,2,3,4,6-penta-O-galloyl-beta-D-glucose (test group), the expression of type I procollagen was increased compared to the group treated with UV light alone (see FIG. 8).

Also, it could be seen that the expression of MMP-1, an enzyme of degrading type I procollagen, increased by UV light, but this increase in expression was inhibited by 1,2,3,4,6-penta-O-galloyl-beta-D-glucose (see FIG. 9).

From the above test results, it was found that 1,2,3,4,6-penta-O-galloyl-beta-D-glucose inhibited the reduction in type I procollagen and the increase in MMP-1 expression, caused by UV light.

### Application Example 1

### Skin Photoaging

Skin photoaging occurs because harmful oxygen species are formed in the human body together with harmful factors of an external environment due to chronic exposure to UV light, and thus the lipid component of the cell membrane is peroxidized and the cell membrane is hardened, so that the intracellular supply of oxygen and nutrients is not smooth. Due to this cause, skin metabolism and skin cell turn-over become slow and, at the same time, the skin epidermal layer becomes thicker, collagen in the skin dermal layer is broken, and elastic fiber elastin is also modified with a reduction in hyaluronic acid as a matrix, resulting in a reduction in skin firmness. This leads to a series of skin photoaging phenomena, including skin dryness in which the skin looks dull and dark and becomes dry and rough, firmness loss, wrinkle formation, weakening of capillary vessel walls, and senile pigmentation. Accordingly, in order to prevent these skin symptoms, 1,2,3,4,6-penta-O-galloyl-beta-D-glucose of the present invention, which inhibits the expression of MMP-1 as collagenase and increases the synthesis of type I procollagen, is effective for the treatment of skin photoaging.

### Application Example 2

### Skin Inflammation

Inflammation refers to the response of living vascular tissue to local injury and is induced because the expression of COX-2 is increased due to active oxygen species produced by UV light. It was reported that changes caused by inflammation include an increase in blood flow, an increase in the permeability of vascular walls, and the infiltration of white blood cells, and cell adhesion molecules are involved in all such changes (Jackson, J. R. et al. FASEB J. 11:457-465, 1997). When inflammation is induced, the infiltration of inflammatory cells is increased in histological terms, and the activity of collagenase is markedly increased in biochemical terms. Accordingly, 1,2,3,4,6-penta-O-galloyl-beta-D-glucose of the present invention, which inhibits the expression of COX-2 that is involved in the progression of inflammatory reactions, is effective for the treatment of inflammation.

### Application Example 3

### Skin Cancer

Skin cancer refers to all types of cancers occurring on the skin, and is divided, according to the kind of skin on which it occurred, into basal cell carcinoma, squamous cell carcinoma, melanoma and the like. The basal cell carcinoma is the most common malignant tumor occurring in the human beings, including the skin. The most important inducer thereof is UV light and can occur in sites where traumatic injuries, scars or burns have been present. In skin symptoms, one or several small nodes become gradually larger and, at the same time, the center thereof is depressed, the portion thereof forms an ulcer or a scab, and the peripheral portion thereof is slightly protruded in the form of a bank. Because it is associated mainly with UV light, it occurs on the face, particularly on the nose, forehead, ear and cheek.

Squamous cell carcinoma is a primary malignant tumor common next to basal cell carcinoma The important cause of squamous cell carcinoma is UV light, like the case of basal cell carcinoma, and other causes of squamous cell carcinoma include genetic factors, various chemical substances known as carcinogens, radiation, papilloma virus, scars caused by burns, and the like. In symptoms thereof, it often appears on sites exposed to UV light, including the face or hand and is in the form of a hard tumor, a scab covers the surface thereof, and an ulcer appears after the scab is detached.

Accordingly, 1,2,3,4,6-penta-O-galloyl-beta-D-glucose of the present invention, which inhibits the expression of collagenase MMP-1 that is increased due to UV light, and inhibits the expression of COX-2 that is overexpressed in skin cancer tissue, is effective for the treatment of skin cancer.

<Preparation 1>

Preparation of Agent Comprising Pharmaceutical Composition for Preventing or Treating Skin Disease

<1-1> Preparation of Tablets

25 mg of 1,2,3,4,6-penta-O-galloyl-beta-D-glucose of the present invention, 26 mg of lactose, an excipient for direct tableting, 3.5 mg of Avicel (microcrystalline cellulose), 1.5 mg of sodium starch glycolate, a disintegrant, and 8 mg low-hydroxypropylcellulose (L-HPC) for direct tableting, a binder, were mixed with each other in an U-shaped mixer for 20 minutes. After completion of mixing, the mixture was added with 1 mg of magnesium stearate, a lubricant, and further mixed for 3 minutes. Through a quantitative test and a constant humidity test, the mixture was then tableted and film-coated to obtain tablets.

<1-2> Preparation of Syrup

A syrup containing 2% (w/v) of 1,2,3,4,6-penta-O-galloyl-beta-D-glucose of the present invention or its pharmaceutically acceptable salt as an active ingredient was prepared in the following manner: 2 g of an acid addition salt of 1,2,3,4,6-penta-O-galloyl-beta-D-glucose of the present invention, 0.8 g of saccharin, and 25.4 g of sugar were dissolved in 80 g of hot water. The solution was cooled, to which 8.0 g of glycerin, 0.04 g of a flavoring agent, 4.0 g of ethanol, 0.4 g of sorbic acid, and a suitable amount of distilled water were then added. The mixture was added with water to a total volume of 100 ml.

<1-3> Preparation of Capsules

50 mg of 1,2,3,4,6-penta-O-galloyl-beta-D-glucose of the present invention, 50 mg of lactose, 46.5 mg of starch, 1 mg of talc, and a suitable amount of magnesium stearate were mixed with each other. The mixture was packed into hard gelatin capsules to obtain capsules.

<1-4> Preparation of Injection Solution

An injection solution containing 10 mg of 1,2,3,4,6-penta-O-galloyl-beta-D-glucose was prepared in the following manner: 1 g of a hydrochloride salt of 1,2,3,4,6-penta-O-galloyl-beta-D-glucose of the present invention, 0.6 g of sodium chloride, and 0.1 g of ascorbic acid were dissolved in distilled water to prepare 100 ml of a solution. The solution was poured in a bottle and sterilized by heating for 30 minutes at 20° C.

<1-5> Preparation of Cream Composition

To 40 g of a cream base containing oily components, aqueous components and surfactants, including 1.5 g of stearic acid, 2.2 g of stearyl alcohol, 0.5 g of butyl stearate, 0.5 g of propylene glycol, 2.0 g of glycerin monostearate and 0.3 g of potassium hydroxide, 1,2,3,4,6-penta-O-galloyl-beta-D-glucose of the present invention was added at a concentration of 2% (w/w). The mixture was emulsified in a mixer, degassed, filtered and cooled, thus preparing a cream composition. To the composition, a chelating agent, perfume and a pigment were added as additives, and the composition was formulated in the form of an oil-in-water emulsion, such that it contained a small amount of oily components.

### INDUSTRIAL APPLICABILITY

As can be seen from the foregoing, 1,2,3,4,6-penta-O-galloyl-beta-D-glucose of the present invention is effective against various skin diseases, because it has the activities of inhibiting a reduction in type I procollagen, an increase in MMP-1 expression, an increase in COX-2 expression, wrinkle formation, epidermal thickening, and collagen degradation. Accordingly, 1,2,3,4,6-penta-O-galloyl-beta-D-glucose of the present invention is useful for the prevention and treatment of skin diseases.

Although the preferred embodiment of the present invention has been described for illustrative purposes, those skilled in the art will appreciate that various modifications, additions and substitutions are possible, without departing from the scope and spirit of the invention as disclosed in the accompanying claims.

1. A pharmaceutical composition for preventing and treating skin disease, comprising 1,2,3,4,6-penta-O-galloyl-beta-D-glucose represented by formula 1 or pharmaceutically acceptable salt thereof as an active ingredient:

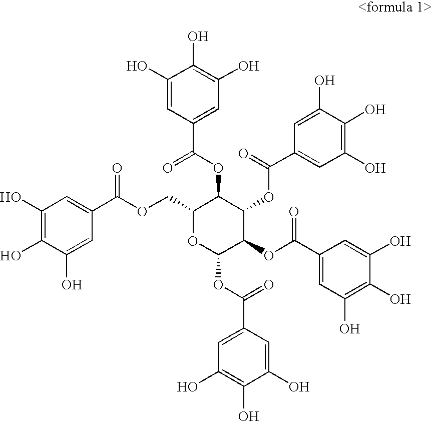

2. The pharmaceutical composition according to claim 1, wherein the skin disease is skin disease caused by ultraviolet radiation, or inflammatory skin disease.

3. The pharmaceutical composition according to claim 2, wherein the skin disease caused by ultraviolet radiation is photo aging.

4. The pharmaceutical composition according to claim 2, wherein the inflammatory skin disease is selected from the group consisting of skin inflammation, acute and chronic eczema, contact dermatitis, atopic dermatitis, seborrheic dermatitis, lichen simplex chronicus, intertrigo, dermatitis exfoliativa, papular urticaria, psoriasis, solar dermatitis and acne.

5. A cosmetic composition for preventing or improving skin disease, comprising 1,2,3,4,6-penta-O-galloyl-beta-D-glucose represented by formula 1 or salt thereof:

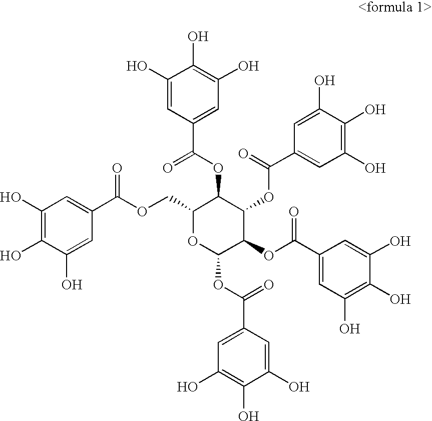

6. A method for treating or preventing skin disease, comprising administrating to a subject in need thereof an effective amount of 1,2,3,4,6-penta-O-galloyl-beta-D-glucose or pharmaceutically acceptable salt thereof.

7. The method according to claim 6, wherein the skin disease is skin disease caused by ultraviolet radiation, or inflammatory skin disease.

8. Use of 1,2,3,4,6-penta-O-galloyl-beta-D-glucose or pharmaceutically acceptable salt thereof for the manufacture of an agent for treating skin disease.

9. The use according to claim 8, wherein the skin disease is skin disease caused by ultraviolet radiation, or inflammatory skin disease.

10. Use of 1,2,3,4,6-penta-O-galloyl-beta-D-glucose or salt thereof for the manufacture of a cosmetic agent for preventing or improving skin disease.

11. The use according to claim 10, wherein the skin disease is skin disease caused by ultraviolet radiation, or inflammatory skin disease.
